(19) United States
(12) Patent Application Publication
(10) Pub. No.: US 2019/0254353 A1
(43) Pub. Date: Aug. 22, 2019

(54) SHEATH Dual Pouch Underwear

(71) Applicant: Robert Michael Patton, Colorado Springs, CO (US)

(72) Inventor: Robert Michael Patton, Colorado Springs, CO (US)

(21) Appl. No.: 15/899,274

(22) Filed: Feb. 19, 2018

(51) Int. Cl. A41B 9/02; A41B 9/00; A61F 2/26

(52) U.S. Cl. CPC A41B 9/023; A41B 9/001; A41B 2400/20; A61F 2/26; A41B 9/026

(57) ABSTRACT

Dual Pouch Underwear for use by all humans is underwear or undergarment specifically designed to create anatomical separation of male parts (real or prosthetic). The larger pouch separates the testicles from the inner thighs and perineum, while the circular opening/pouch separates the penis head and shaft from the testicles. The larger pouch also creates light support by cupping the testicle and scrotum, rather than letting the testicles and scrotum hang freely. The circular opening/pouch is covered by fabric which has a fly opening to facilitate urination. In addition, the circular opening/pouch provides a layer of protection from urine splash, fecal matter, and other elements from contacting the rest of the genitalia while urinating. The separation of both the testicle and the shaft, limit skin to skin contact therefore keeping those wearing the dual pouch underwear, cooler, less sweaty, more comfortable, and less susceptible to skin irritation.

### FIELD OF THE INVENTION

This invention relates to underwear and undergarments generally, and in-particular to underwear and undergarment having a dual pouch, separating the testicles from inner thighs as well as separating the penis head and shaft from the testicles.

### BACKGROUND OF THE INVENTION

The idea behind the original SHEATH Underwear design was conceived in August of 2008 by US Army Veteran, Robert Patton. At the time of conception, Patton was actively serving in the armed forces and was stationed in the Iraqi desert for his second tour of the Iraq war—his first being from June 2006 to January 2007. All too familiar with the sweltering heat and unforgiving conditions of the middle-eastern region, and the discomfort that comes with it, Patton used the little spare time that he had to devise possible methods for effectively combating the moisture and irritation that plagues those who are subjected to these types of environments. This is what lead him to look at the point of contact between a man and his clothing—his underwear.

As the war began to take a peaceful turn and entered a state of decline, Patton utilized the last of his deployment time as a chance to contemplate potential designs and meet with local clothiers and tailors to weigh his fabric and seaming options. About two years and a handful of prototypes later, Patton received his first marketable shipment of SHEATH Underwear 1.0s in September of 2010, and thus marked the official launch of the SHEATH apparel brand and the foundation from which we continue to innovate new variations of our original design i.e. SHEATH Underwear 2.0, 3.0, Dual-Pouch, et cetera.

### BRIEF SUMMARY OF THE INVENTION

The invention of the dual pouch underwear system creates an anatomical separation of the testicles from the perineum and inner thighs, and the shaft and head of penis from testicles through the design of a large pouch for the testicles, combined with an additional smaller outer pouch for the penis shaft. Once the wearer is in both pouches, the fabric separates the anatomy, decreases skin to skin contact, and allows air flow throughout the genital region.

### BRIEF DESCRIPTION OF THE DRAWINGS

FIG. 1 is a frontal view of the garment 8.

FIG. 2 is an inside-out frontal view of the garment 8 showing the dual pouch technology.

FIG. 3 is a right perspective view of the garment 8.

FIG. 4 is a right perspective view of an inside-out version of the of the garment 8 showing the dual pouch technology.

FIG. 5 is a top view of the garment 8.

FIG. 6 is an undershot view of the garment 8.

FIG. 7 is an inside-out frontal view featuring a demonstration 48 of the functionality of the dual pouch as indicated in FIG. 2.

### DETAILED DESCRIPTION OF THE INVENTION

The invention of the dual pouch underwear system solves a multitude of problems not addressed by more typical underwear found in everyday stores. More specifically, the dual pouch system creates an anatomical separation of the testicles from the perineum and inner thighs, and the shaft and head of penis from testicles. The dual pouch system accomplishes this through the design of a larger pouch for the testicles, combined with an additional smaller outer pouch accessed via circular seam for the penis shaft. Once the wearer is in both pouches, the fabric separates the anatomy, decreases skin to skin contact, and allows air flow throughout the genital region. In the case of pre-op transgender wearers, the pouch allows prosthetics to be separated and look more natural to the eye.

The descriptions of the drawings in the next section will provide further detail regarding the functionality and design of the dual pouch system/underwear.

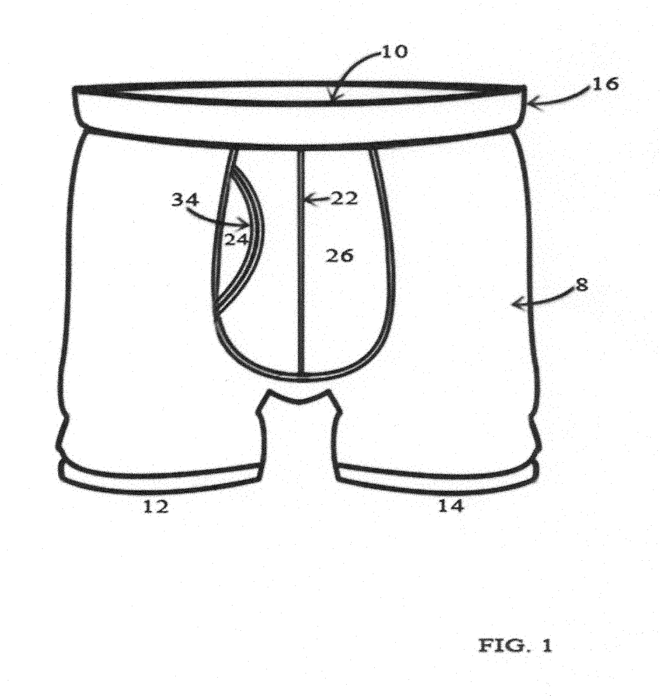

FIG. 1: A frontal view of the 8 garment as depicted in the figure, having an opening 10 for the trunk and opening 12 for right leg and 14 for left leg. An elastic waistband 16 is sewn into the material of the waist. The intricately placed ventilation seam 34 for added convenience is connected to the outermost layer 26 of fabric in the dual pouch system which covers the middle layer as depicted in drawing 24.

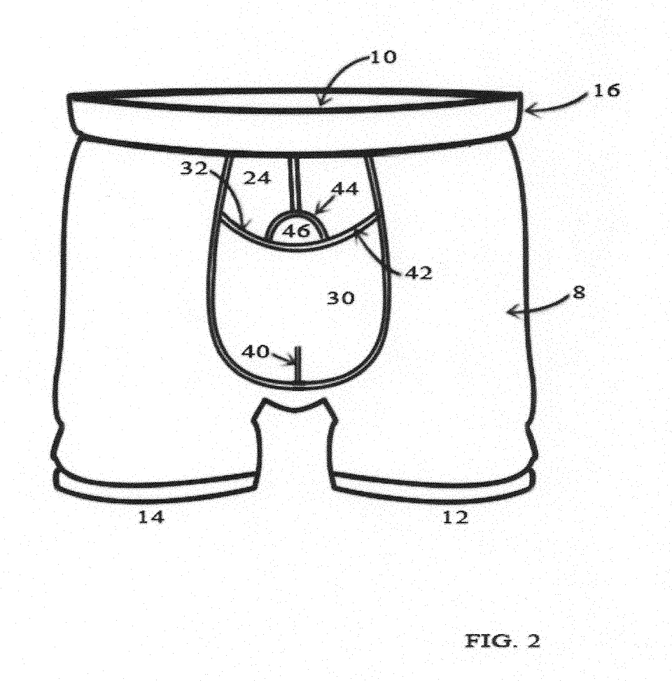

FIG. 2: Inside out frontal view of the garment showing the dual pouch technology. The fabric 30 and seam 42 of the main isolation pouch provides separation from testicles to inner thigh region. The vertical seam at the base of the pouch 40 not only provides support but volume to the inside of the pouch 32. The second of the dual pouch system 46, which isolates the penis from the testicles is supported by the circular elastic seam 44 sewn into the middle layer 24 of the pouch The left leg 14 and right leg 12 respectively appear to be reversed from FIG. 1 because the garment 8 is inside out. The Waist Band 16 is inside out and the opening for trunk 10 is still in the same spot.

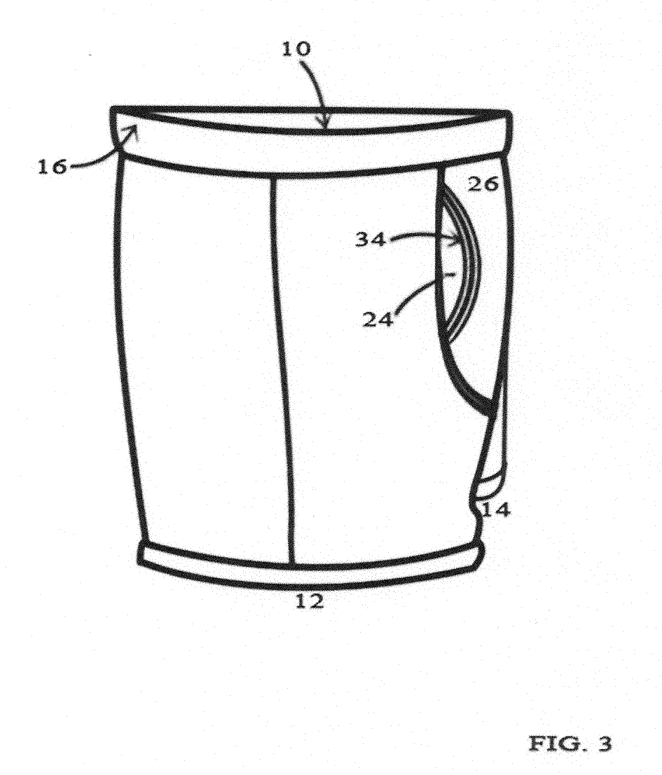

FIG. 3: A view of the garment 8 shown from the right perspective emphasizing the ventilation seam 34 and the middle panel 24 and showing the overall outside of the garment 8 such as the right leg 12 left leg 14 and waistband 10 are depicted in the figure.

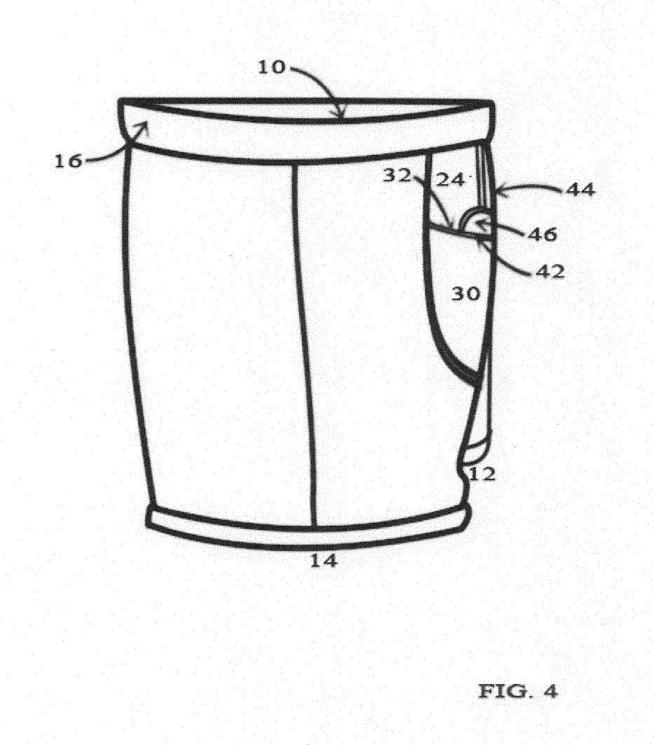

FIG. 4: An inside out version of the right side perspective illustrating right 12 and left legs 14 openings trunk opening 10 and waistband 16 showing the dual pouch technology as follows. The fabric 30 and seam 42 of the main isolation pouch 32 provides separation from testicles to inner thigh region. The second layer of the dual pouch system 46, which isolates the penis from the testicles is supported by the circular elastic seam 44 sewn into the middle layer 24 of the pouch. This ensures separation of the penis from testicles.

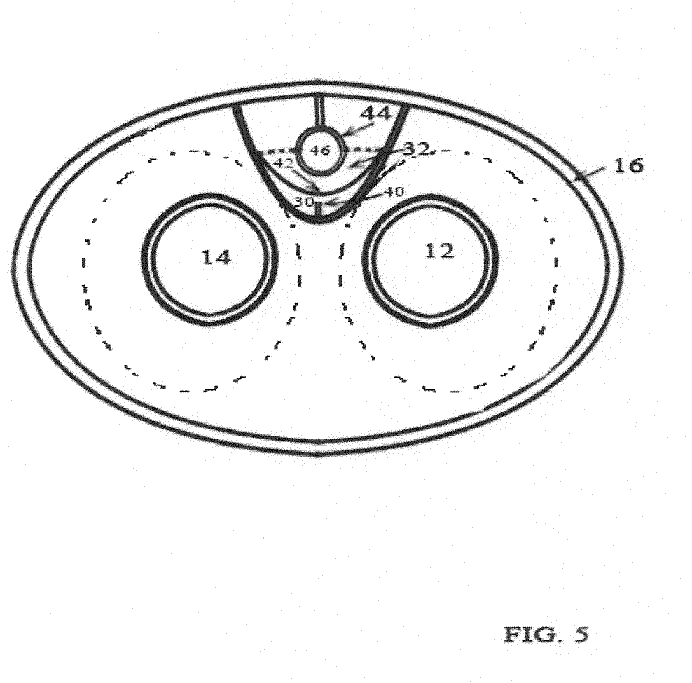

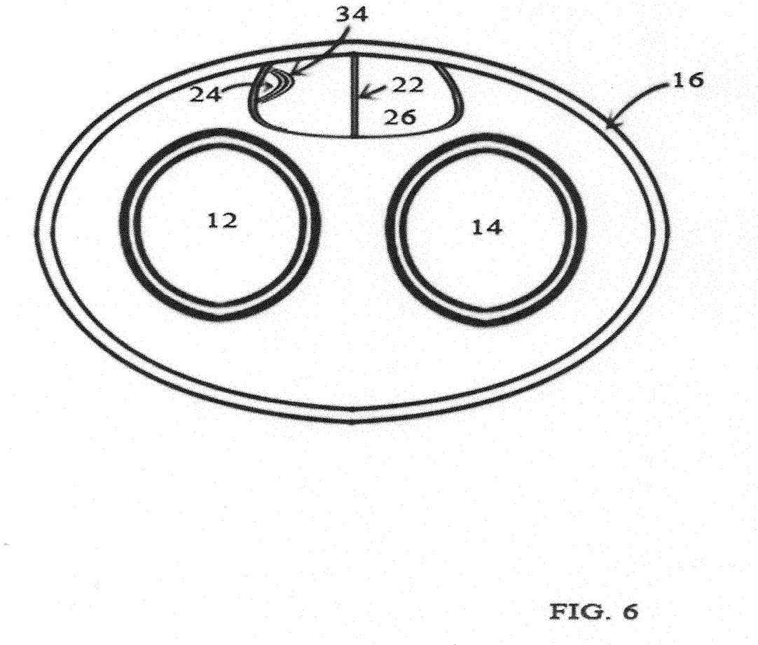

FIG. 5: An aerial view showing the inside functionality of the product, emphasizing separation as indicated in FIG. 2. The fabric 30 and seam 42 of the main isolation pouch provides separation from testicles to inner thigh region. The vertical seam at the base of the pouch 40 not only provides support but volume to the inside of the pouch 32. The second of the dual pouch system 46, which isolates the penis from the testicles is supported by the circular elastic seam 44. The left leg 14 and right leg 12 respectively appear to be reversed from FIG. 6 because the garment is inside out. The Waist Band 16 is indicated and shown inside out.

FIG. 6: An undershot perspective view of the garment from underneath, showing the ventilation seam 34 sewn into the outer layer 26 which exposes the middle layer 24. Additionally the waistband 16 is illustrated with the right leg 12 and left leg 14 hole openings to indicate proper perspective.

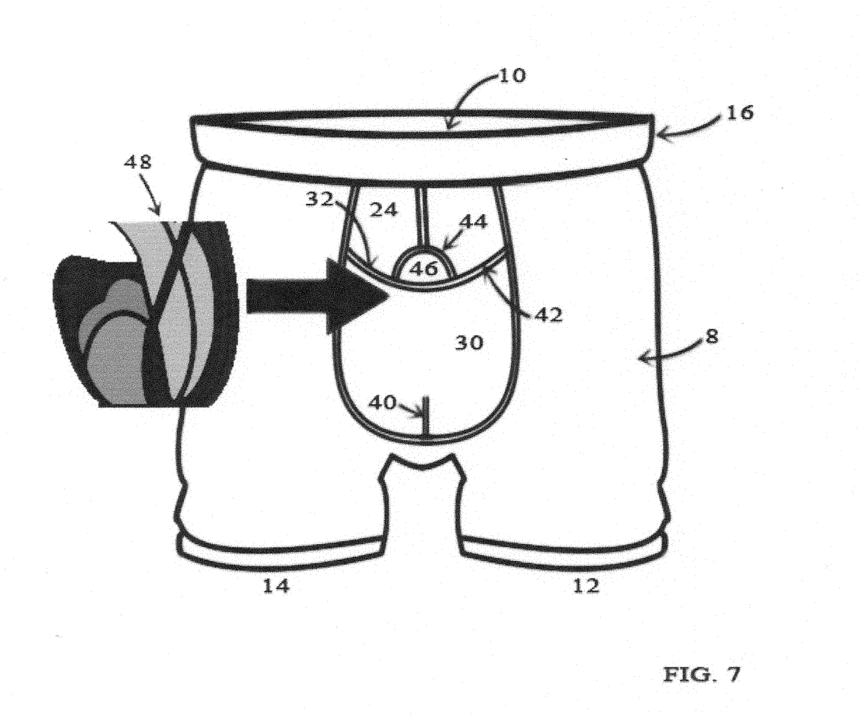

FIG. 7: Frontal inside out view featuring a demonstration 48 of the functionality of the dual pouch as indicated in FIG. 2. The fabric 30 and seam 42 of the main isolation pouch provides separation from testicles to inner thigh region. The vertical seam at the base of the pouch 40 not only provides support but volume to the inside of the pouch 32. The second of the dual pouch system 46, which isolates the penis from the testicles is supported by the circular elastic seam 44 sewn into the middle layer 24 of the pouch. The left leg 14 and right leg 12 respectively appear to be reversed from FIG. 1 because the garment 8 is inside out. The Waist Band 16 is inside out and the opening for trunk 10 is still in the same spot.

1. An article of clothing comprising:
a. Cloth underwear having openings for right leg, left leg, and trunk;
b. Elastic waistband;
c. Dual pouch inside underwear comprising: consisting of
i. Cloth pouch inside underwear for testicles; and
ii. Circular opening/pouch on front side of underwear for penis head and shaft; and
d. Cloth fly cover with opening for urination and ventilation.

2. The article of clothing of claim 1, wherein the dual pouch creates separation of testicles from inner thigh and perineum, and wherein when testicles are placed inside of cloth pouch, cloth is positioned between the testicles and legs, and between the testicles and perineum.

3. The article of clothing of claim 2 wherein separation of penis head and shaft from testicles is achieved when user places penis shaft and head in circular opening/pouch cloth.

4. The article of clothing of claim 2, wherein the cloth pouch creates light support reducing scrotal fatigue by cupping the testicle and scrotum.

5. The article of clothing of claim 1 wherein the dual pouch is configured to accept prosthetic male anatomy parts.

6. The article of clothing of claim 1, wherein the cloth fly comprises two cloth pieces sewn with smooth seam in center of fly opening, creating convex shape AND providing room for male anatomy.

7. The article of clothing of claim 2, wherein the cloth allows air in between the skin of the testicles, legs, and perineum, decreasing sweat and skin sticking together.

8. The article of clothing of claim 3, wherein the cloth allows air in between the skin of the testicles and the skin of the penis shaft and head, decreasing sweat and skin sticking together.

9. A dual pouch garment, comprising:
pieces of cloth attached together to form a torso opening and two leg openings;
an elastic waistband attached around the opening for torso;
a first pouch configured for placement of testicles therein, the first pouch formed by attaching a first pouch-forming cloth to an interior side of the garment at a front lower portion between the two leg openings; and
a second pouch formed by a middle cloth layer having an opening for placement of a penis therethrough and an outer cloth layer.

10. The dual pouch garment of claim 9, wherein the opening for placement of the penis therethrough comprises a circular elastic seam sewn into the middle cloth layer.

11. The dual pouch garment of claim 9, wherein the outer cloth layer provides a fly opening configured to facilitate urination.

12. The dual pouch garment of claim 9, wherein the first pouch includes a vertical seam sewn at a base portion of the first pouch-forming cloth.
